(19) United States
(12) Patent Application Publication
(10) Pub. No.: US 2024/0062439 A1
(43) Pub. Date: Feb. 22, 2024

(54) DISPLAY PROCESSING APPARATUS, METHOD, AND PROGRAM

(71) Applicant: FUJIFILM Corporation, Tokyo (JP)

(72) Inventor: Takuya TSUTAOKA, Kanagawa (JP)

(73) Assignee: FUJIFILM Corporation, Tokyo (JP)

(21) Appl. No.: 18/495,787

(22) Filed: Oct. 27, 2023

Related U.S. Application Data
(63) Continuation of application No. PCT/JP2022/014343, filed on Mar. 25, 2022.

(30) Foreign Application Priority Data
May 6, 2021 (JP) 2021-078434

(51) Int. Cl. G06T 11/20; G06T 7/13; G06V 10/764; G06T 7/11

(52) U.S. Cl. CPC G06T 11/203; G06T 7/13; G06V 10/764; G06T 7/11; G06V 2201/031; G06T 2207/10132; G06T 2207/10068; G06T 2207/30092; G06T 2207/20081

(57) ABSTRACT

Provided are a display processing apparatus, method, and program for displaying a region of a detection target object in an image in a manner intelligible to a user even if the contour or boundary of the detection target object is unclear. A transmitting/receiving unit (100) and an image generation unit (102), which function as an image acquisition unit, perform an image acquisition process for sequentially acquiring ultrasound images. A region extraction unit (106) extracts a rectangular region including an organ, which is a detection target object, from an acquired ultrasound image. A curve generation unit (108) generates, in the extracted rectangular region, a curve corresponding to the organ in the rectangular region. An image combining unit (109) combines the ultrasound image and the generated curve corresponding to the organ. A display control unit (110) causes a monitor (18) to display the ultrasound image combined with the curve.

### CROSS-REFERENCE TO RELATED APPLICATIONS

The present application is a Continuation of PCT International Application No. PCT/JP2022/014343 filed on Mar. 25, 2022 claiming priority under 35 U.S.C § 119(a) to Japanese Patent Application No. 2021-078434 filed on May 6, 2021. Each of the above applications is hereby expressly incorporated by reference, in its entirety, into the present application.

### BACKGROUND OF THE INVENTION

### 1. Field of the Invention

The present invention relates to a display processing apparatus, method, and program, and more specifically to a technique for drawing and displaying a region of a detection target object detected from an image.

### 2. Description of the Related Art

A medical image diagnostic apparatus including this type of function has been proposed in JP-H6-233761A.

The medical image diagnostic apparatus described in JP-H6-233761A includes target region extraction means for roughly extracting an intended site in an image, a neural network that receives an image of the extracted target region and predicts broad-view information for recognizing the intended site, intended site recognition means for recognizing the intended site by using the broad-view information predicted by the neural network and outputting data thereof (the contour of the intended site), and an image display unit that receives data of a recognition result output from the intended site recognition means and displays the recognition result together with an original image.

The intended site recognition means detects the contour of the intended site by detecting, at respective equal angular pitches from the origin S (x0, y0) of the broad-view information, the positions of all points whose density values change from “0” to “1” toward the outside of the image, finding a boundary between black and white binary values of “1” and “0” by using the broad-view information as a guide, and extracting the contour as the intended site.

### SUMMARY OF THE INVENTION

The broad-view information predicted by the neural network described in JP-H6-233761A indicates region information of a rough intended site, and guides detection of the positions of all points (contour points) whose density values change from “0” to “1” toward the outside of the image at respective equal angular pitches from the origin of the broad-view information such that the contour points do not greatly deviate from the broad-view information.

That is, the medical image diagnostic apparatus described in JP-H6-233761A, which tracks the contour of an intended site on the basis of a change in density value, uses broad-view information as a guide to help trace the boundary.

However, JP-H6-233761A does not describe how to determine contour points, when searching for contour points whose density values change from “0” to “1” toward the outside of the image at the respective equal angular pitches, in a case where no contour point is found in the vicinity of the broad-view information. In particular, there is a tendency not to find contour points in a case where the contour or boundary of an intended site in the image is unclear.

If no contour point is found, points on a rough contour indicated by the broad-view information may be used. However, in this case, contour locations in which contour points are found and rough contour locations indicated by the broad-view information due to no contour points found are mixed together, and the contour of the intended site to be extracted may be unnatural.

The present invention has been made in view of such circumstances, and an object thereof is to provide a display processing apparatus, method, and program for displaying a region of a detection target object in an image in a manner intelligible to a user even if the contour or boundary of the detection target object is unclear.

To achieve the object described above, according to a first aspect, the invention provides a display processing apparatus including a processor. In the display processing apparatus, the processor is configured to perform an image acquisition process for acquiring an image; a region extraction process for extracting a region including a detection target object from the acquired image; a curve generation process for generating, in the extracted region, a curve corresponding to the detection target object in the region; an image combining process for combining the image and the curve; and a display process for causing a display device to display the image combined with the curve.

According to the first aspect of the present invention, a region including a detection target object is extracted, and, in the extracted region, a curve corresponding to the detection target object in the region is generated. Thus, a curve to be generated can be generated without deviating from the region including the detection target object, and generated as a curve having a small deviation from the actual contour of the detection target object. Further, the generated curve is combined with the image and is displayed on the display device. This makes it possible to display the region of the detection target object in a manner intelligible to the user.

In a display processing apparatus according to a second aspect of the present invention, preferably, the region extraction process extracts a rectangular region as the region. This is because extraction of a rectangular region including the detection target object from the image enables robust learning and estimation of the detection target object even if the contour of the detection target object is partially unclear.

In a display processing apparatus according to a third aspect of the present invention, preferably, the curve generation process generates the curve in accordance with a predetermined rule.

In a display processing apparatus according to a fourth aspect of the present invention, preferably, the curve generation process selects a first template curve from a plurality of template curves prepared in advance, and deforms the first template curve to fit the region to generate the curve.

In a display processing apparatus according to a fifth aspect of the present invention, preferably, the processor is configured to perform a class classification process for classifying the detection target object into a class on the basis of the image, and the curve generation process selects the first template curve from the plurality of template curves on the basis of a classification result of the class classification process. The detection target object has an outer shape corresponding to the classified class. Accordingly, selecting a first template curve from among the plurality of template curves on the basis of a classification result obtained by classifying the detection target object into a class makes it possible to select a first template curve suitable for the detection target object.

In a display processing apparatus according to a sixth aspect of the present invention, preferably, the curve generation process selects the first template curve by selecting one template curve from the plurality of template curves and deforming the selected template curve to fit the region, selection of the first template curve being based on a distribution of pixel values in an inner region and a distribution of pixel values in an outer region, the inner region and the outer region being obtained by dividing the region into the inner region and the outer region by using the deformed template curve.

In a display processing apparatus according to a seventh aspect of the present invention, preferably, the curve generation process deforms the first template curve to fit at least one of a size or an aspect ratio of the region to generate the curve.

In a display processing apparatus according to an eighth aspect of the present invention, preferably, the curve generation process deforms the first template curve so as to increase a difference between a distribution of pixel values in an inner region and a distribution of pixel values in an outer region, the inner region and the outer region being obtained by dividing the region into the inner region and the outer region by using the template curve.

In a display processing apparatus according to a ninth aspect of the present invention, preferably, the curve generation process generates the curve by using one parametric curve or using a plurality of parametric curves in combination. A B -spline curve, a Bezier curve, or the like can be applied as a parametric curve.

In a display processing apparatus according to a tenth aspect of the present invention, preferably, the curve generation process adjusts a parameter of the one parametric curve or the plurality of parametric curves so as to increase a difference between a distribution of pixel values in an inner region and a distribution of pixel values in an outer region, the inner region and the outer region being obtained by dividing the region into the inner region and the outer region by using the one parametric curve or the plurality of parametric curves.

In a display processing apparatus according to an eleventh aspect of the present invention, preferably, the curve generation process extracts a plurality of points having a large gradient of pixel values in the region and adjusts a parameter of the one parametric curve or the plurality of parametric curves by using the plurality of points as control points.

In a display processing apparatus according to a twelfth aspect of the present invention, preferably, the curve generation process performs image processing on pixel values in the region and extracts a contour of the detection target object to generate the curve.

In a display processing apparatus according to a thirteenth aspect of the present invention, preferably, the curve generation process determines, for each section of the generated curve, whether the section has a typical pixel value therearound, and deletes a section other than a section having the typical pixel value while leaving the section having the typical pixel value undeleted. A section that is a section of the generated curve and that includes a large number of typical pixel values (for example, a section with little noise and relatively uniform pixel values) is considered to be the contour of the detection target object and is thus left undeleted, and the other sections are deleted as sections corresponding to an unclear contour.

In a display processing apparatus according to a fourteenth aspect of the present invention, preferably, the curve generation process deletes a section other than at least one of a section having a large curvature or a section including an inflection point in the generated curve while leaving the at least one section undeleted. This is because the curves of the other sections, which are other than a section having a large curvature and a section around an inflection point, are close to a straight line and thus the contour of the detection target object can be inferred even if such curves are deleted. If the section to be deleted is excessively long, a proportion of the section is preferably left undeleted.

In a display processing apparatus according to a fifteenth aspect of the present invention, preferably, a plurality of different rules are prepared, and the processor is configured to perform a class classification process for classifying the detection target object into a class on the basis of the image and select a rule to be used to generate the curve from the plurality of different rules in accordance with a classification result of the class classification process.

In a display processing apparatus according to a sixteenth aspect of the present invention, preferably, the image is an ultrasound image. In the ultrasound image, typically, the contour or boundary of a detection target object in the image is unclear and difficult to identify. Thus, the ultrasound image is effective as an image to which the display processing apparatus according to the present invention is applied. The ultrasound image also includes an endoscopic ultrasound image captured with an ultrasonic endoscope apparatus.

In a display processing apparatus according to a seventeenth aspect of the present invention, the detection target object is an organ.

According to an eighteenth aspect, the invention provides a display processing method performed by a processor. The display processing method includes a step of acquiring an image, a step of extracting a region including a detection target object from the acquired image, a step of generating, in the extracted region, a curve corresponding to the detection target object in the region, a step of combining the image and the curve, and a step of causing a display device to display the image combined with the curve.

According to a nineteenth aspect, the invention provides a display processing program for causing a computer to implement a function of acquiring an image, a function of extracting a region including a detection target object from the acquired image, a function of generating, in the extracted region, a curve corresponding to the detection target object in the region, a function of combining the image and the curve, and a function of causing a display device to display the image combined with the curve.

According to the present invention, a region of a detection target object in an image can be displayed in a manner intelligible to a user even if the contour or boundary of the detection target object is unclear.

### BRIEF DESCRIPTION OF THE DRAWINGS

FIG. 1 is a schematic diagram illustrating an overall configuration of an ultrasonic endoscope system including a display processing apparatus according to the present invention.

FIG. 2 is a block diagram illustrating an embodiment of an ultrasonic processor device that functions as the display processing apparatus according to the present invention;

FIG. 3 is a diagram illustrating an example of an ultrasound image on which a rectangular frame enclosing an organ is superimposed;

FIG. 4 is a diagram used to describe a first embodiment of a curve generation process performed by a curve generation unit;

FIG. 5 is a diagram used to describe a modification of the first embodiment of the curve generation process performed by the curve generation unit;

FIG. 6 is a diagram used to describe a second embodiment of the curve generation process performed by the curve generation unit;

FIG. 7 is a diagram used to describe a third embodiment of the curve generation process performed by the curve generation unit;

FIGS. 8A and 8B are diagrams used to describe a fourth embodiment of the curve generation process performed by the curve generation unit;

FIGS. 9A and 9B are diagrams used to describe a fifth embodiment of the curve generation process performed by the curve generation unit; and

FIG. 10 is a flowchart illustrating an embodiment of a display processing method according to the present invention.

### DESCRIPTION OF THE PREFERRED EMBODIMENTS

Preferred embodiments of a display processing apparatus, method, and program according to the present invention will be described hereinafter with reference to the accompanying drawings.

### Overall Configuration of Ultrasonic Endoscope System Including Display Processing Apparatus

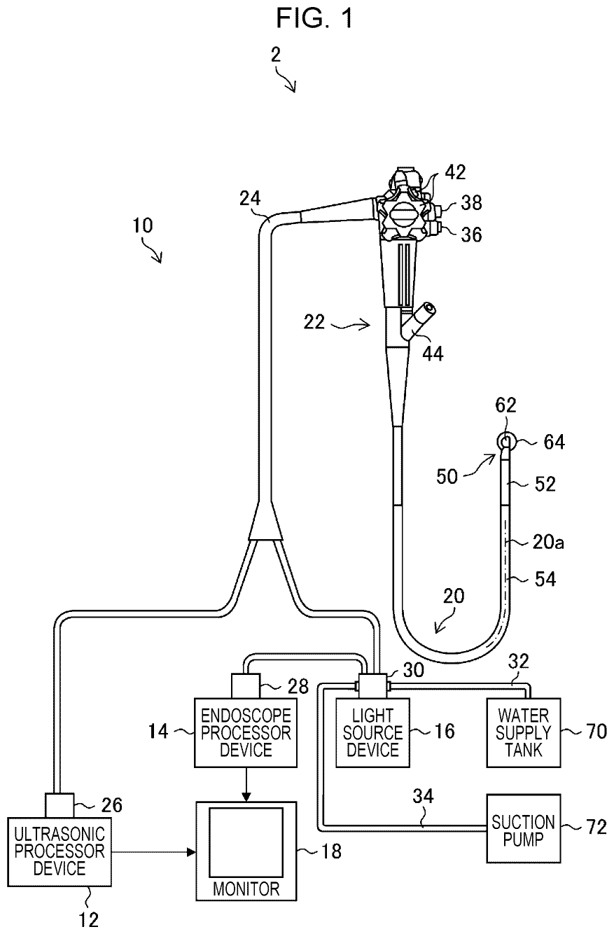

FIG. 1 is a schematic diagram illustrating an overall configuration of an ultrasonic endoscope system including a display processing apparatus according to the present invention.

As illustrated in FIG. 1, an ultrasonic endoscope system 2 includes an ultrasound scope 10, an ultrasonic processor device 12 that generates an ultrasound image, an endoscope processor device 14 that generates an endoscopic image, a light source device 16 that supplies illumination light to the ultrasound scope 10 to illuminate the inside of a body cavity, and a display device (monitor) 18 that displays the ultrasound image and the endoscopic image.

The ultrasound scope 10 includes an insertion section 20 to be inserted into a body cavity of a subject, a handheld operation section 22 coupled to a proximal end portion of the insertion section 20 and to be operated by an operator, and a universal cord 24 having one end connected to the handheld operation section 22. The other end of the universal cord 24 is provided with an ultrasonic connector 26 to be connected to the ultrasonic processor device 12, an endoscope connector 28 to be connected to the endoscope processor device 14, and a light source connector 30 to be connected to the light source device 16.

The ultrasound scope 10 is detachably connected to the ultrasonic processor device 12, the endoscope processor device 14, and the light source device 16 through the connectors 26, 28, and 30, respectively. The light source connector 30 is also connected to an air/water supply tube 32 and a suction tube 34.

The monitor 18 receives respective video signals generated by the ultrasonic processor device 12 and the endoscope processor device 14 and displays an ultrasound image and an endoscopic image. The ultrasound image and the endoscopic image can be displayed such that only one of the images is appropriately switched and displayed on the monitor 18 or both of the images are simultaneously displayed.

The handheld operation section 22 is provided with an air/water supply button 36 and a suction button 38, which are arranged side by side, and is also provided with a pair of angle knobs 42 and a treatment tool insertion port 44.

The insertion section 20 has a distal end, a proximal end, and a longitudinal axis 20a. The insertion section 20 is constituted by a tip main body 50, a bending part 52, and an elongated long flexible soft part 54 in this order from the distal end side of the insertion section 20. The tip main body 50 is formed by a hard member. The bending part 52 is coupled to the proximal end side of the tip main body 50. The soft part 54 couples the proximal end side of the bending part 52 to the distal end side of the handheld operation section 22. That is, the tip main body 50 is disposed on the distal end side of the insertion section 20 in the direction of the longitudinal axis 20a. The bending part 52 is remotely operated to bend by turning the pair of angle knobs 42 disposed in the handheld operation section 22. As a result, the tip main body 50 can be directed in a desired direction.

The tip main body 50 is attached with an ultrasound probe 62 and a bag-like balloon 64 that covers the ultrasound probe 62. The balloon 64 can expand or contract when water is supplied from a water supply tank 70 or the water in the balloon 64 is sucked by a suction pump 72. The balloon 64 is inflated until the balloon 64 abuts against the inner wall of the body cavity to prevent attenuation of an ultrasound wave and an ultrasound echo (echo signal) during ultrasound observation.

The tip main body 50 is also attached with an endoscopic observation portion (not illustrated) having an illumination portion and an observation portion including an objective lens, an imaging element, and so on. The endoscopic observation portion is disposed behind the ultrasound probe 62 (on the handheld operation section 22 side).

An ultrasound image acquired by the ultrasonic endoscope system 2 or the like includes speckle noise. In the ultrasound image, the contour or boundary of a detection target object in the image is unclear and difficult to identify, and this tendency is noticeable in a portion near a signal region. Accordingly, a large organ such as the pancreas, which is depicted over the entire signal region, has a problem in that, in particular, the contour of the organ is difficult to accurately estimate.

Known approaches with AI (Artificial Intelligence) for an expected scene include the following two approaches: (1) region extraction (semantic segmentation) and (2) object detection.

The approach (1) is an approach for causing the AI to classify the pixels in an image to determine which organ each pixel belongs to. In this approach, it can be expected to acquire an accurate organ map. However, a drawback is that learning and estimation are unstable for an organ whose contour is partially unclear.

The approach (2) is an approach for causing the AI to estimate a minimum region (rectangular region) containing each organ. In this approach, it is possible to robustly learn and estimate an organ whose contour is partially unclear. However, a drawback is that, for an organ (such as the pancreas) depicted as being elliptical or bean-shaped, the deviation of the contour of the organ from the estimated rectangular region is large and it is difficult for a user to understand a specific organ position if a rectangular frame (bounding box) indicating the rectangular region is displayed as it is.

Another drawback is that, when a plurality of organs to be detected are adjacent to each other or the organs have an inclusion relationship, if detection results are displayed as bounding boxes, the bounding boxes overlap each other, resulting in very low visibility.

The present invention has overcome the drawbacks of the approach (2) and provides a display processing apparatus that displays the position of a detection target object (organ) whose contour is unclear in a manner intelligible to a user. Since the problem of an unclear object or contour is likely to occur in a typical image or the like captured in a dark place prone to insufficient exposure, the present invention can also be applied to images other than an ultrasound image.

### Display Processing Apparatus

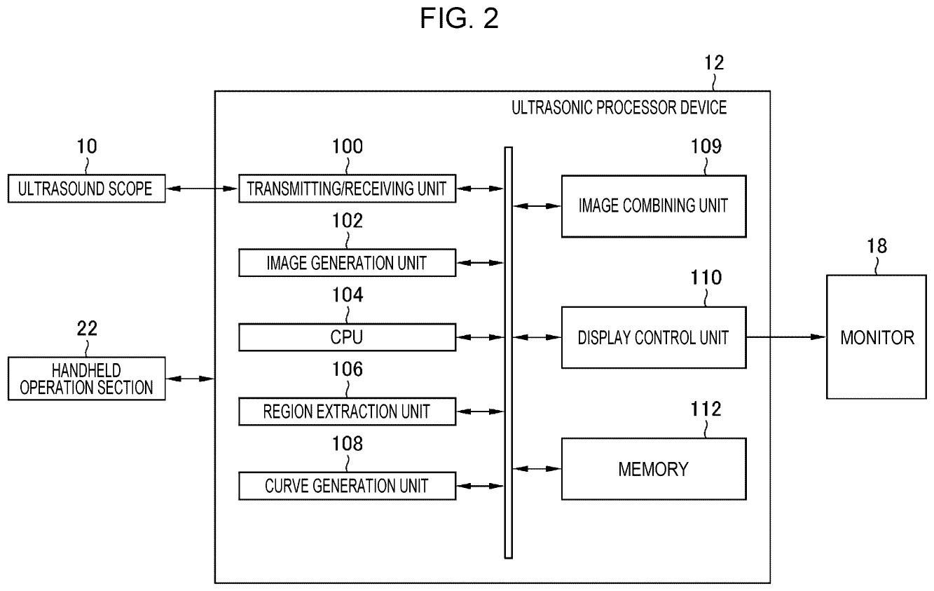

FIG. 2 is a block diagram illustrating an embodiment of an ultrasonic processor device that functions as a display processing apparatus according to the present invention.

The ultrasonic processor device 12 illustrated in FIG. 2 is configured to generate, based on sequentially acquired images (in this example, ultrasound images), curves corresponding to the contours of detection target objects (in this example, various organs) in an image, combine the generated curves with the image to a composite image, and cause the monitor 18 to display the composite image to support a user in observing the image.

The ultrasonic processor device 12 illustrated in FIG. 2 includes a transmitting/receiving unit 100, an image generation unit 102, a CPU (Central Processing Unit) 104, a region extraction unit 106, a curve generation unit 108, an image combining unit 109, a display control unit 110, and a memory 112. The processing of each unit is implemented by one or more processors.

The CPU 104 operates in accordance with various programs stored in the memory 112 and including a display processing program according to the present invention to perform overall control of the transmitting/receiving unit 100, the image generation unit 102, the region extraction unit 106, the curve generation unit 108, the image combining unit 109, and the display control unit 110. Further, the CPU 104 functions as some of these units.

The transmitting/receiving unit 100 and the image generation unit 102, which function as an image acquisition unit, are portions that perform an image acquisition process for sequentially acquiring ultrasound images.

A transmitting unit of the transmitting/receiving unit 100 generates a plurality of drive signals to be applied to a plurality of ultrasonic transducers of the ultrasound probe 62 of the ultrasound scope 10, assigns respective delay times to the plurality of drive signals on the basis of a transmission delay pattern selected by a scan control unit (not illustrated), and applies the plurality of drive signals to the plurality of ultrasonic transducers.

A receiving unit of the transmitting/receiving unit 100 amplifies a plurality of detection signals, each of which is output from one of the plurality of ultrasonic transducers of the ultrasound probe 62, and converts the detection signals from analog detection signals to digital detection signals (also referred to as RF (Radio Frequency) data). The RF data is input to the image generation unit 102.

The image generation unit 102 assigns respective delay times to the plurality of detection signals represented by the RF data on the basis of a reception delay pattern selected by the scan control unit and adds the detection signals together to perform reception focus processing. Through the reception focus processing, sound ray data in which the focus of the ultrasound echo is narrowed is formed.

The image generation unit 102 further corrects the sound ray data for attenuation caused by the distance in accordance with the depth of the reflection position of the ultrasound wave by using STC (Sensitivity Time Control), and then performs envelope detection processing on the corrected sound ray data by using a low pass filter or the like to generate envelope data. The image generation unit 102 stores envelope data for one frame or more preferably for a plurality of frames in a cine memory (not illustrated). The image generation unit 102 performs pre-process processing, such as Log (logarithmic) compression and gain adjustment, on the envelope data stored in the cine memory to generate a B-mode image.

In this way, the transmitting/receiving unit 100 and the image generation unit 102 acquire time-series B-mode images (hereafter referred to as “images”).

The region extraction unit 106 is a portion that performs a region extraction process for extracting, based on an input image, a region (in this example, a “rectangular region”) including a detection target object in the image. For example, the region extraction unit 106 can be implemented by AI.

In this example, the detection target object is any organ in ultrasound images (tomographic images of B-mode images), and examples of such an organ include the pancreas, the main pancreatic duct, the spleen, the splenic vein, the splenic artery, and the gallbladder.

When images, each being of one frame of a moving image, are sequentially input, the region extraction unit 106 performs a region extraction process for detecting (recognizing) one or more organs in each of the input images and extracting (estimating) a region including the organ(s). The region including the organ(s) is a minimum rectangular region containing the organ(s).

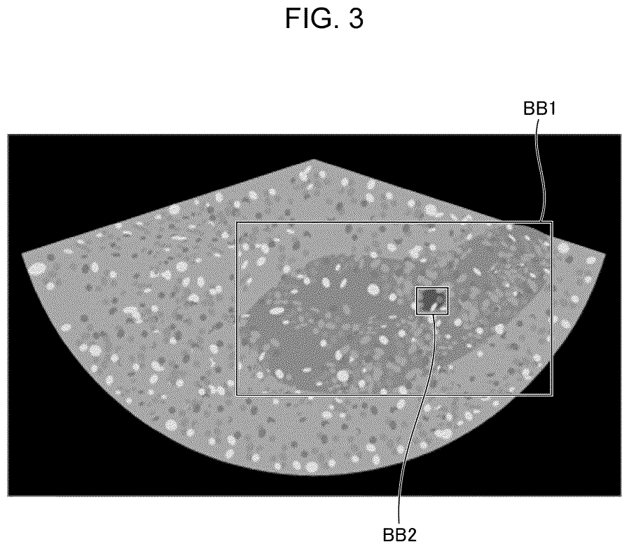

FIG. 3 is a diagram illustrating an example of an ultrasound image on which a rectangular frame enclosing an organ is superimposed.

In the example illustrated in FIG. 3, a rectangular frame (bounding box) BB1 indicating a rectangular region containing an organ includes the pancreas, and a bounding box BB2 includes the main pancreatic duct.

The region extraction unit 106 may also perform a classification process for classifying the detection target object into any one of a plurality of classes on the basis of an input image. As a result, the type of each organ serving as the detection target object can be recognized, and a name or abbreviation indicating the type of the organ can be displayed in association with the corresponding organ.

Referring back to FIG. 2, the curve generation unit 108 is a portion that performs a curve generation process on the rectangular region extracted by the region extraction unit 106 to generate a curve corresponding to the detection target object in the rectangular region.

The curve generation unit 108 performs the curve generation process in accordance with a predetermined rule, which will be described below.

### First Embodiment of Curve Generation Process

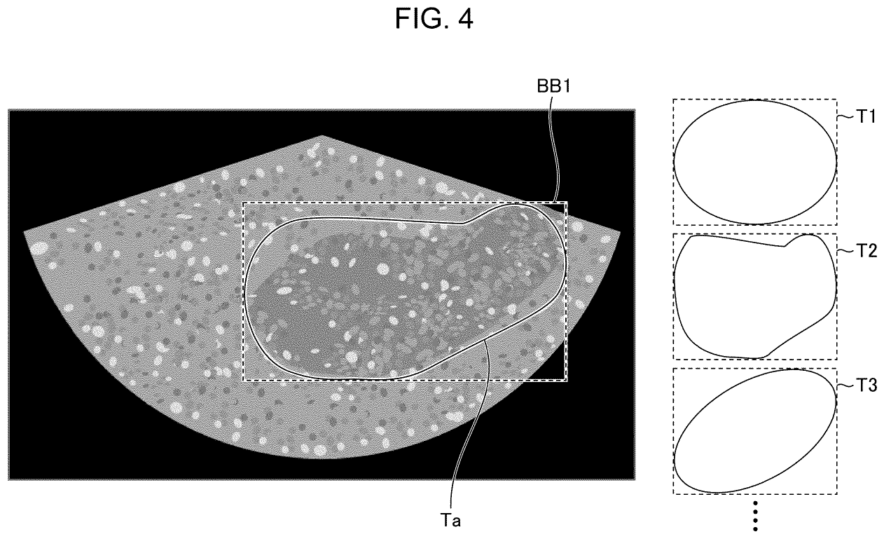

FIG. 4 is a diagram used to describe a first embodiment of a curve generation process performed by the curve generation unit.

The memory 112 illustrated in FIG. 2 stores a plurality of template curves T1, T2, T3, . . . , which are prepared in advance. Template curves having shapes such as a circular shape, an elliptical shape, and a bean shape are prepared as the plurality of template curves.

In the first embodiment of the curve generation process performed by the curve generation unit 108, a first template curve is selected from the plurality of template curves prepared in advance.

In the selection of the first template curve, the first template curve can be selected from the plurality of template curves on the basis of a classification result obtained by classifying an organ serving as a detection target object into a class.

This is because the shape of the organ has a shape corresponding to the classification result obtained by classifying the organ (that is, the type of the organ) into a class.

The detection target object can be classified into a class by the region extraction unit 106 having the AI function or the CPU 104 classifying the pixels in the input image to determine which class (which organ) each pixel belongs to.

In another selection of the first template curve, as described below, which template curve matches the detection target object is determined by actual application. That is, one template curve Ti (i=1, 2, 3, . . . ) is selected from the plurality of template curves T1, T2, T3, . . . , and the selected template curve Ti is deformed to fit the rectangular region (the bounding box BB1). When the selected template curve Ti. is deformed to fit the rectangular region, the rectangular region is divided into an inner region and an outer region by the deformed template curve Ti. A difference between the distribution of pixel values in the inner region and the distribution of pixel values in the outer region is used to select a first template curve.

Preferably, a template curve Ti for which the difference between the distribution of pixel values in the inner region and the distribution of pixel values in the outer region is largest is selected as the first template curve. Alternatively, a template curve Ti for which the difference exceeds a threshold value may be selected as the first template curve. Alternatively, the first template curve may be selected by combining the above-described method using the classification result obtained by class classification and a method of determining whether a template curve matches the detection target object through actual application. For example, a plurality of template curves serving as candidates for the first template curve may be extracted from the plurality of template curves on the basis of a classification result obtained by class classification, and whether the plurality of extracted template curves match the detection target object may be actually applied to select the first template curve.

Upon selection of the first template curve in the way described above, the curve generation unit 108 deforms the first template curve to fit the first template curve in the rectangular region. For example, the curve generation unit 108 deforms the selected first template curve to fit at least one of the size or aspect ratio of the rectangular region to generate a curve corresponding to the detection target object.

In the example illustrated in FIG. 4, a template curve T2, which is suitable for the shape of the pancreas serving as a detection target object, is selected as the first template curve. The template curve T2 is deformed so as to be inscribed in the bounding box BB 1 to generate a curve Ta corresponding to the detection target object.

### Modification of First Embodiment of Curve Generation Process

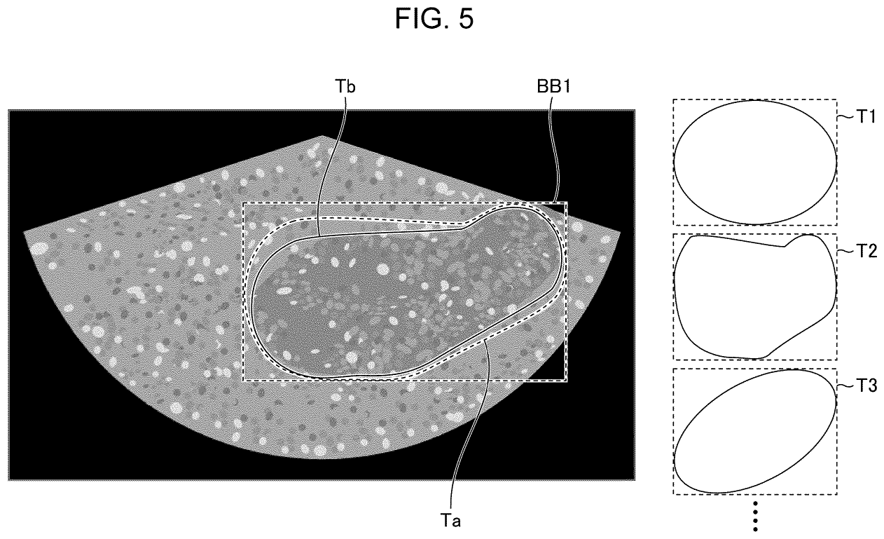

FIG. 5 is a diagram used to describe a modification of the first embodiment of the curve generation process performed by the curve generation unit.

As illustrated in FIG. 5, the curve generation unit 108 further deforms the curve Ta, which is generated by the first embodiment of the curve generation process illustrated in FIG. 4, to generate a curve Tb corresponding to the detection target object.

Specifically, the rectangular region of the bounding box BB 1 is divided into an inner region and an outer region by the curve Ta, which is simply deformed so as to be inscribed in the bounding box BB 1. The curve generation unit 108 further deforms the curve Ta so as to increase the difference between the distribution of pixel values in the inner region and the distribution of pixel values in the outer region to generate the curve Tb.

The curve Tb generated in this way can be closer to the contour of the pancreas serving as the detection target object than the curve Ta obtained by simply deforming the template curve T2.

### Second Embodiment of Curve Generation Process

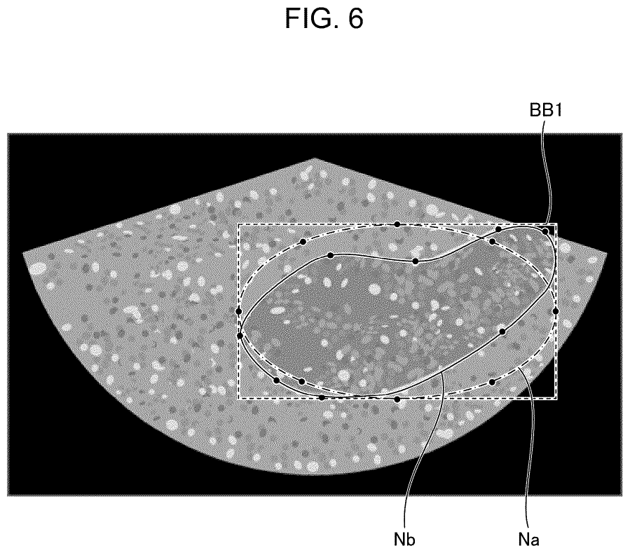

FIG. 6 is a diagram used to describe a second embodiment of the curve generation process performed by the curve generation unit.

The memory 112 illustrated in FIG. 2 stores a plurality of parametric curves prepared in advance. Possible examples of the plurality of parametric curves include a spline curve and a Bezier curve. Examples of the spline curve include an N-th order spline curve, a B-spline curve, and a NURBS (Non-Uniform Rational B-Spline) curve. The NURBS curve is a generalized curve of a B-spline curve. The Bezier curve is an (N−1)-th order curve obtained from N control points, and is a special case of a B-spline curve.

The curve generation unit 108 uses one parametric curve or a plurality of parametric curves in combination to generate a curve corresponding to the detection target object.

In the example illustrated in FIG. 6, the curve generation unit 108 generates a NURBS curve Na formed in an ellipse inscribed in the bounding box BB1. The NURBS curve Na passes through eight control points on the ellipse.

The curve generation unit 108 changes parameters of the NURBS curve Na and searches for a state that best fits the contour of the pancreas serving as the detection target object to generate a final curve Nb corresponding to the detection target object.

That is, the region of the bounding box BB1 is divided into an inner region and an outer region by a parametric curve. The curve generation unit 108 adjusts parameters of the parametric curve so as to increase the difference between the distribution of pixel values in the inner region and the distribution of pixel values in the outer region to generate the curve Nb, which best fits the contour of the detection target object.

### Third Embodiment of Curve Generation Process

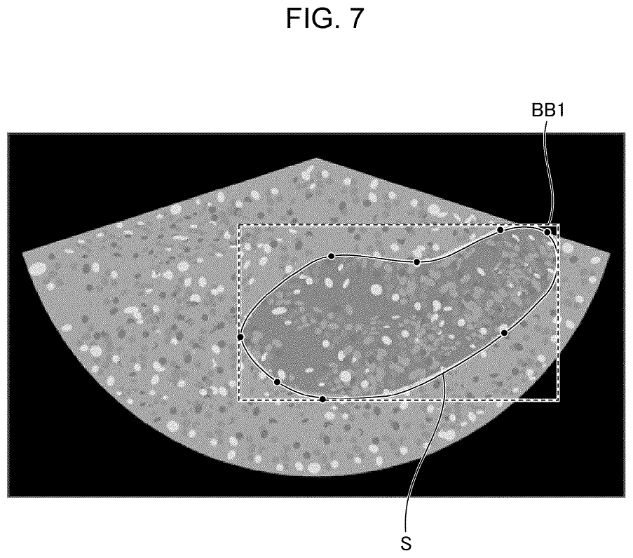

FIG. 7 is a diagram used to describe a third embodiment of the curve generation process performed by the curve generation unit.

The third embodiment of the curve generation process performed by the curve generation unit 108 is similar to the second embodiment illustrated in FIG. 6 in that a parametric curve is used, but determines a plurality of control points in advance to determine parameters of the parametric curve.

As illustrated in FIG. 7, the curve generation unit 108 searches for a plurality of points (control points) having a large luminance gradient within the bounding box BB1. The number of control points is three or more to form a closure. In the example illustrated in FIG. 7, eight control points are determined.

The curve generation unit 108 uses these control points to adjust the parameters of the parametric curve. That is, for example, the curve generation unit 108 generates a three-dimensional spline curve S passing through the control points. Thereafter, the curve generation unit 108 changes the position or number of control points to search for the most suitable state and determines the three-dimensional spline curve S. Suitability can be determined by using, for example, the difference between the distributions of pixel values inside and outside the three-dimensional spline curve S.

The generated curve need not pass through any control point when the curve is a B-spline curve.

### Fourth Embodiment of Curve Generation Process

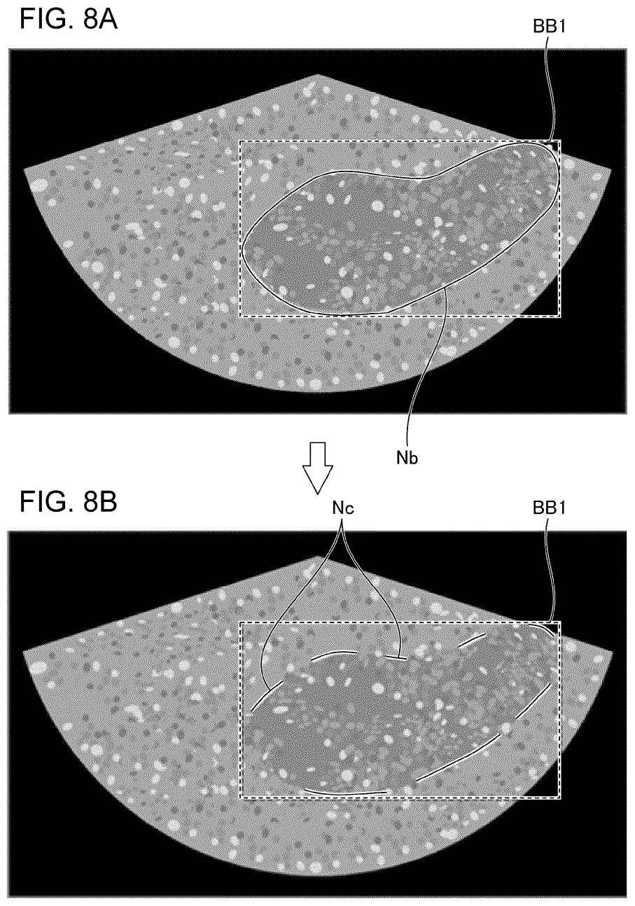

FIGS. 8A and 8B are diagrams used to describe a fourth embodiment of the curve generation process performed by the curve generation unit.

In the fourth embodiment of the curve generation process performed by the curve generation unit 108, as illustrated in FIG. 8A, a curve Nb corresponding to the contour of the detection target object is generated. The curve Nb can be generated by, for example, the embodiments illustrated in FIGS. 5 to 7.

Then, as illustrated in FIG. 8B, the curve generation unit 108 determines, for each section of the generated curve Nb, whether the section has typical pixel values therearound. The curve generation unit 108 deletes sections other than sections Nc having typical pixel values while leaving the sections Nc undeleted.

That is, the curve generation unit 108 refers to, for each of the points on the generated curve Nb (FIG. 8A), pixel values in a neighboring region inside and outside the curve, and deletes sections other than the sections Nc including a large number of typical pixel values (for example, sections with little noise and relatively uniform pixel values) while leaving the sections Nc undeleted (FIG. 8B).

As a result, a section considered to be the contour of a detection target object can be left undeleted, and a section corresponding to an unclear contour can be deleted.

### Fifth Embodiment of Curve Generation Process

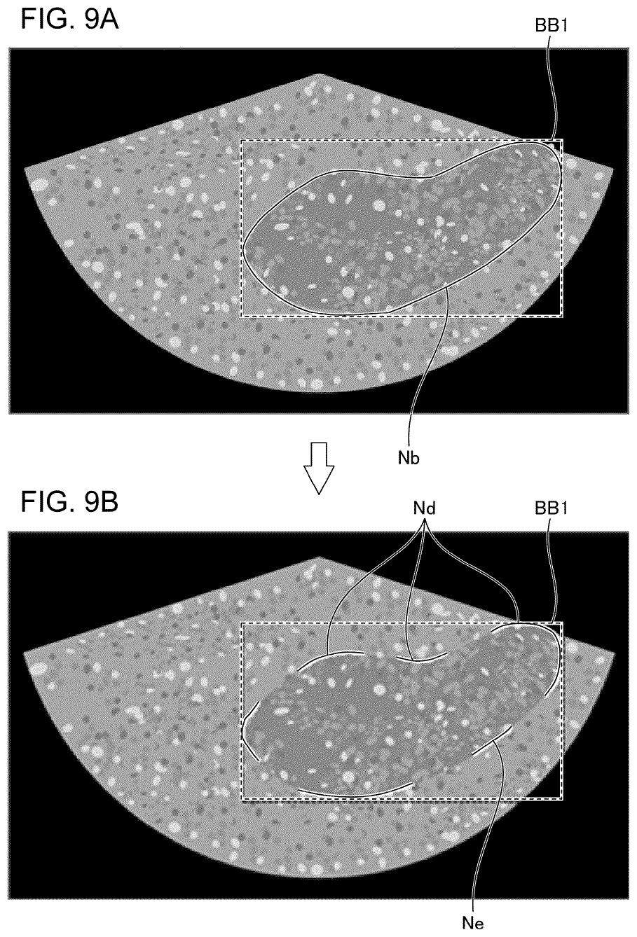

FIGS. 9A and 9B are diagrams used to describe a fifth embodiment of the curve generation process performed by the curve generation unit.

In the fifth embodiment of the curve generation process performed by the curve generation unit 108, as illustrated in FIG. 9A, a curve Nb corresponding to the contour of the detection target object is generated. The curve Nb can be generated by, for example, the embodiments illustrated in FIGS. 5 to 7.

Then, as illustrated in FIG. 9B, the curve generation unit 108 leaves, for sections of the generated curve Nb, sections Nd of the generated curve Nb undeleted, each of the sections Nd being at least one of a section having a large curvature or a section including an inflection point, and deletes the other sections.

This is because the curves of the other sections, which are other than a section having a large curvature and a section around an inflection point, are close to a straight line and thus the contour of the detection target object can be inferred even if such curves are deleted. If the section to be deleted is excessively long, a proportion of the section is preferably left undeleted. In FIG. 9B, a section Ne is a section that is left undeleted because the section to be deleted is excessively long.

The curve generation process under a predetermined rule is not limited to those in the first to fifth embodiments, and may perform image processing on pixel values in a rectangular region and extract the contour of the detection target object to generate a curve.

For example, the contour of the detection target object is extracted by using an edge extraction filter (for example, a Sobel filter) having a size sufficiently larger than the size of speckle noise to prevent the extraction of the contour of the detection target object from being affected by the speckle noise. The edge extraction filter may be used to scan the rectangular region, and edges (contour points) of the detection target object may be extracted from scan positions at which the output value of the edge extraction filter exceeds a threshold value. The extracted contour points are joined together. As a result, a curve can be generated even if some of the contour points of the detection target object fail to be detected.

While the first to fifth embodiments represent embodiments of a curve generation process under respective predetermined rules, it is preferable to appropriately select which rule to use to generate a curve in accordance with the class classification of the detection target object.

That is, as typified by the first to fifth embodiments, a plurality of different rules for the curve generation process are stored in the memory 112. The CPU 104 performs a class classification process for classifying the detection target object into a class on the basis of the image, and selects a rule to be used to generate a curve from the plurality of different rules stored in the memory 112 in accordance with a classification result of the class classification process. The curve generation unit 108 performs the curve generation process in accordance with the selected rule.

When a plurality of detection target objects are present in one image, a rule is selected for each of the detection target objects, and a curve corresponding to each detection target object is generated in accordance with the selected rule.

Referring back to FIG. 2, the image combining unit 109 performs an image combining process for combining the image acquired and generated by the image generation unit 102 and the like and the curve generated by the curve generation unit 108. The curve is different in luminance or color from nearby portions and is combined as a line drawing having a line width that is visible to the user.

The display control unit 110 causes the monitor 18 to display images that are sequentially acquired by the transmitting/receiving unit 100 and the image generation unit 102 and with which the curve corresponding to the detection target object, which is generated by the curve generation unit 108, is combined. In this example, the display control unit 110 causes the monitor 18 to display a moving image indicating an ultrasound tomographic image.

Each of FIGS. 4 to 7, 8B, and 9B illustrates a state in which a curve (solid line) corresponding to the detection target object is displayed to be superimposed on the image. However, unlike FIGS. 6 and 7, no control point is displayed.

This makes it possible to display a region of a detection target object in an image in a manner intelligible to a user even if the contour or boundary of the detection target object is unclear.

While the bounding box BB1 indicated by a broken line is not displayed in this example, the display control unit 110 may display the bounding box BB1. Alternatively, if information on a class into which the detection target object is classified is acquired, the display control unit 110 may display text information indicating the class obtained by classification (for example, text information of an abbreviation or formal name of the type of the organ) in association with the detection target object.

### Display Processing Method

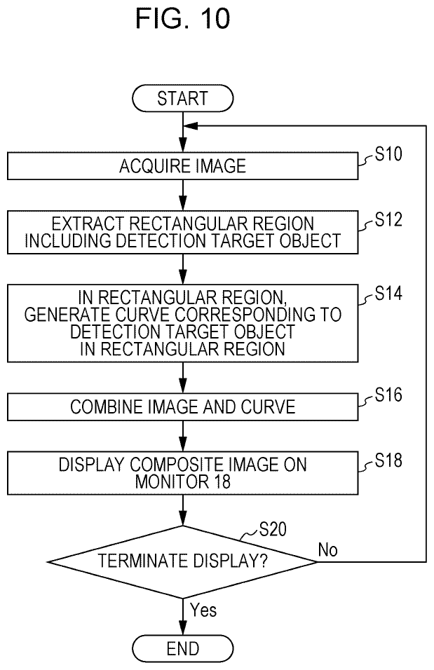

FIG. 10 is a flowchart illustrating an embodiment of a display processing method according to the present invention, and illustrates a processing procedure of the units of the ultrasonic processor device 12 illustrated in FIG. 2.

In FIG. 10, the transmitting/receiving unit 100 and the image generation unit 102, which function as an image acquisition unit, acquire time-series images (step S10). For example, in the case of time-series images with a frame rate of 30 fps (frames per second), an image for one frame is acquired every 1/30 (seconds).

Then, the region extraction unit 106 recognizes, based on an image acquired in step S10, a detection target object (organ) present in the image, and extracts a rectangular region including the organ (step S12).

Then, in the rectangular region extracted by the region extraction unit 106, the curve generation unit 108 generates a curve corresponding to the detection target object in the rectangular region (step S14). The process of generating the curve corresponding to the detection target object includes, as described above, a method using a template curve, a method using a parametric curve, and so on (see FIGS. 4 to 9B), and detailed description thereof will be omitted.

The image combining unit 109 combines the image acquired in step S10 and the curve generated in step S14 (step S16). The display control unit 110 causes the monitor 18 to display an image combined with the curve in step S16 (step S18).

This allows the user to easily check a region of a detection target object in an image even if the contour or boundary of the detection target object is unclear.

Then, the CPU 104 determines whether to terminate the display of the time-series B-mode images in accordance with the user's operation (Step S20).

If it is determined that the display of the images is not to be terminated (in the case of “No”), the process returns to step S10, and the processing of steps S10 to S20 is repeated for the image of the next frame. If it is determined that the display of the images is to be terminated (in the case of “Yes”), the display process ends.

### Others

In the present embodiment, the ultrasonic processor device 12 includes a function as a display processing apparatus according to the present invention. However, the present invention is not limited thereto, and a personal computer or the like separate from the ultrasonic processor device 12 may acquire an image from the ultrasonic processor device 12 and function as a display processing apparatus according to the present invention.

The present invention is not limited to an ultrasound image, and can further be applied to a still image rather than a moving image. Further, the detection target object in the image is not limited to various organs and may be, for example, a lesion region.

The hardware structure for performing various kinds of control of the ultrasonic processor device (display processing apparatus) of the present embodiment is implemented as various processors as described as follows. The various processors include a CPU (Central Processing Unit), which is a general-purpose processor executing software (program) to function as various control units, a programmable logic device (PLD) such as an FPGA (Field Programmable Gate Array), which is a processor whose circuit configuration is changeable after manufacture, a dedicated electric circuit, which is a processor having a circuit configuration specifically designed to execute specific processing, such as an ASIC (Application Specific Integrated Circuit), and so on.

A single control unit may be configured by one of the various processors or by a combination of two or more processors of the same type or different types (for example, a plurality of FPGAs or a combination of a CPU and an FPGA). Alternatively, a plurality of control units may be configured by a single processor. Examples of configuring a plurality of control units by a single processor include, first, a form in which, as typified by a computer such as a client or server computer, the single processor is configured by a combination of one or more CPUs and software and the processor functions as the plurality of control units. The examples include, second, a form in which, as typified by a system on chip (SoC) or the like, a processor is used in which the functions of the entire system including the plurality of control units are implemented by a single IC (Integrated Circuit) chip. As described above, the various control units are configured using one or more of the various processors described above as a hardware structure.

The present invention further includes a display processing program to be installed in a computer to cause the computer to function as a display processing apparatus according to the present invention, and a non-volatile storage medium having the display processing program recorded thereon.

Furthermore, it goes without saying that the present invention is not limited to the embodiments described above and various modifications may be made without departing from the spirit of the present invention.

### REFERENCE SIGNS LIST

2 ultrasonic endoscope system
10 ultrasound scope
12 ultrasonic processor device
14 endoscope processor device
16 light source device
18 monitor
20 insertion section
20a longitudinal axis
22 handheld operation section
24 universal cord
26 ultrasonic connector
28 endoscope connector
30 light source connector
32 tube
34 tube
36 air/water supply button
38 suction button
42 angle knob
44 treatment tool insertion port
50 tip main body
52 bending part
54 soft part
62 ultrasound probe
64 balloon
70 water supply tank
72 suction pump
100 transmitting/receiving unit
102 image generation unit
104 CPU
106 region extraction unit
108 curve generation unit
109 image combining unit
110 display control unit
112 memory
S10 to S20 step

What is claimed is:

1. A display processing apparatus comprising
a processor configured to perform:
an image acquisition process for acquiring an image;
a region extraction process for extracting a region including a detection target object from the acquired image;
a curve generation process for generating, in the extracted region, a curve corresponding to the detection target object in the region;
an image combining process for combining the image and the curve; and
a display process for causing a display device to display the image combined with the curve, wherein
the region extraction process extracts a rectangular region as the region.

2. The display processing apparatus according to claim 1, wherein
the region extraction process is performed using an AI (Artificial Intelligence), and
the AI receives the acquired image and outputs the rectangular region including the detection target object in the acquired image.

3. The display processing apparatus according to claim 1, wherein
the curve generation process generates the curve in accordance with a predetermined rule.

4. The display processing apparatus according to claim 3, wherein
the curve generation process selects a first template curve from a plurality of template curves prepared in advance, and deforms the first template curve to fit the region to generate the curve.

5. The display processing apparatus according to claim 4, wherein
the processor is configured to perform a class classification process for classifying the detection target object into a class on the basis of the image, and
the curve generation process selects the first template curve from the plurality of template curves on the basis of a classification result of the class classification process.

6. The display processing apparatus according to claim 4, wherein
the curve generation process selects the first template curve by selecting one template curve from the plurality of template curves and deforming the selected template curve to fit the region, selection of the first template curve being based on a distribution of pixel values in an inner region and a distribution of pixel values in an outer region, the inner region and the outer region being obtained by dividing the region into the inner region and the outer region by using the deformed template curve.

7. The display processing apparatus according to claim 4, wherein
the curve generation process deforms the first template curve to fit at least one of a size or an aspect ratio of the region to generate the curve.

8. The display processing apparatus according to claim 4, wherein
the curve generation process deforms the first template curve so as to increase a difference between a distribution of pixel values in an inner region and a distribution of pixel values in an outer region, the inner region and the outer region being obtained by dividing the region into the inner region and the outer region by using the first template curve.

9. The display processing apparatus according to claim 3, wherein
the curve generation process generates the curve by using one parametric curve or using a plurality of parametric curves in combination.

10. The display processing apparatus according to claim 9, wherein
the curve generation process adjusts a parameter of the one parametric curve or the plurality of parametric curves so as to increase a difference between a distribution of pixel values in an inner region and a distribution of pixel values in an outer region, the inner region and the outer region being obtained by dividing the region into the inner region and the outer region by using the one parametric curve or the plurality of parametric curves.

11. The display processing apparatus according to claim 9, wherein
the curve generation process extracts a plurality of points having a large gradient of pixel values in the region and adjusts a parameter of the one parametric curve or the plurality of parametric curves by using the plurality of points as control points.

12. The display processing apparatus according to claim 3, wherein
the curve generation process performs image processing on pixel values in the region and extracts a contour of the detection target object to generate the curve.

13. The display processing apparatus according to claim 1, wherein
the curve generation process determines, for each section of the generated curve, whether the section has a typical pixel value therearound, and deletes a section other than a section having the typical pixel value while leaving the section having the typical pixel value undeleted.

14. The display processing apparatus according to claim 1, wherein
the curve generation process deletes a section other than at least one of a section having a large curvature or a section including an inflection point in the generated curve while leaving the at least one section undeleted.

15. The display processing apparatus according to claim 3, wherein
a plurality of different rules are prepared, and
the processor is configured to perform a class classification process for classifying the detection target object into a class on the basis of the image and select a rule to be used to generate the curve from the plurality of different rules in accordance with a classification result of the class classification process.

16. The display processing apparatus according to claim 1, wherein
the image is an ultrasound image.

17. The display processing apparatus according to claim 16, wherein
the detection target object is an organ.

18. A display processing method performed by a processor, the display processing method comprising:
a step of acquiring an image;
a step of extracting a region including a detection target object from the acquired image;
a step of generating, in the extracted region, a curve corresponding to the detection target object in the region;
a step of combining the image and the curve; and
a step of causing a display device to display the image combined with the curve, wherein
the region extracted in the step of extracting, is a rectangular region.

19. The display processing method according to claim 18, wherein
in the step of extracting, an AI (Artificial Intelligence) receives the acquired image and outputs the rectangular region including the detection target object in the acquired image.

20. A non-transitory, computer-readable tangible recording medium which records thereon a program for causing, when read by a computer, the computer to execute the display processing method according to claim 18.
